(19) United States
(12) Patent Application Publication
(10) Pub. No.: US 2024/0159661 A1
(43) Pub. Date: May 16, 2024

(54) COMPOSITE INFRARED SPECTROSCOPY FOR NUTRITION AND FITNESS

(71) Applicant: COR HEALTH, INC., Poway, CA (US)

(72) Inventors: Robert G. MESSERSCHMIDT, Menlo Park, CA (US); Erik ANDRIES, Rio Rancho, NM (US); Dara ROUHOLIMAN, Gold River, CA (US)

(73) Assignee: COR HEALTH, INC., Poway, CA (US)

(21) Appl. No.: 18/549,641

(22) PCT Filed: Mar. 15, 2022
(86) PCT No.: PCT/US2022/020323

§ 371 Date: Sep. 8, 2023

Related U.S. Application Data
(60) Provisional application No. 63/200,562, filed on Mar. 15, 2021.

(51) Int. Cl. G01N 21/35; G01N 33/483; G16H 10/40

(52) U.S. Cl. CPC G01N 21/35; G01N 33/483; G16H 10/40

(57) ABSTRACT

A system, apparatuses, and methods for assisting a person to improve their health. A person's initial blood sample spectra may be processed to identify components that have been found to reflect the impact of a lifestyle program, without identifying a biomarker in the blood. Each lifestyle program may be associated with its own set of spectra components or channel that indicate the impact of the program on blood spectra. Based on the person's initial blood spectra and the channel associated with each program, a process may generate a recommended set of programs that the person could use to improve their health. This set of programs may be filtered based on a demographic characteristic or characteristics of the person by comparing the set of programs to a ranked list of programs found helpful by people with a similar demographic characteristic.

### CROSS-REFERENCE

This application claims priority to U.S. App. No. 63/200,562, filed on 15 Mar. 2021, entitled “COMPOSITE INFRARED SPECTROSCOPY FOR NUTRITION AND FITNESS.”

The subject matter of the present application is related to U.S. application Ser. No. 14/992,945, filed on Jan. 11, 2016, entitled “Spectroscopic measurements with parallel array detector”, published as US20160123869A1 on May 5, 2016, and PCT/US2019/030052, filed on Apr. 30, 2019, entitled “Systems and methods for blood analysis”, published as WO2019213166, the entire disclosures of which are incorporated herein by reference.

### BACKGROUND

Conventional approaches to the delivery of health care and the treatment of disease are less than ideal in at least some respects. Over three trillion dollars is spent annually on health care in the United States, and much of this money, as well as the time and expertise of medical professionals and the supporting medicines, equipment and facilities, is directed towards reacting to symptoms and medical problems caused by preventable diseases. Or, if not preventable, at least diseases for which the long-term harm could be reduced and better managed. It would be beneficial to have improved ways of measuring and analyzing patient characteristics that would allow preventive measures to be taken, such as modifications in diet, exercise, and habits, as well as other aspects of lifestyle.

Another problem with conventional approaches to providing healthcare is that the paradigm for providing care is based substantially around averages of measurable characteristics of a person, such as height, weight, age, blood type, certain biomarkers, etc. This can lead to ineffective or inefficient healthcare as most people are not average in at least some respects that may impact the efficacy of conventional health care methods. Although efforts have been made to personalize healthcare, the effectiveness of personalized care can be limited to the accuracy and frequency of data available for a given subject. Also, many people wish to improve their energy level, athletic performance, or appearance, and such people could benefit from improved information about their wellness and physical conditioning, even though such people may not need medical care or be at risk of disease.

Work in relation to the present disclosure suggests that prior approaches to health and wellness may have placed more reliance on biomarkers than would be ideal. For example, biomarkers may be more difficult to detect than would be ideal in at least some instances. Also, using biomarker data to generate recommendations or suggest medical treatments may result in increased regulatory compliance requirements that are not needed for many effective health and wellness applications, where such applications may still be beneficial in situations where there is a decreased availability of biomarker data.

Many prior approaches typically rely on identifying and monitoring one or more biomarkers in the blood to evaluate a person's health and determine the impact of exercise or diet on their health. For example, a person's cholesterol or triglyceride level, or other marker may be used to evaluate their health prior to and after participation in a change to their lifestyle. While this approach can be helpful it has disadvantages. These disadvantages include the amount of equipment and processing required to properly measure a level of the biomarker and that such an approach may not be efficient for generating a recommendation to a person as to what specific changes to make to have the greatest likelihood of improving their health.

Work in relation to the present disclosure also suggests that it would be helpful to enable the non-invasive measurement of blood characteristics to reduce people's resistance to blood draws and similar laboratory tests. Further, work in relation to the present disclosure suggests that measurements of spectral data and the determination of specific components of that data may be used to evaluate a person's health and if desired, generate recommendations to a person to make a change in their nutrition, exercise regimen, or other characteristic of their lifestyle in order to improve their health.

In light of the above, there is a need for improved systems, devices, and methods for making and using measurements of a person's blood that can be used to provide meaningful information regarding the health and wellness of the subject.

### SUMMARY

The presently disclosed system, methods, and apparatus provide a way to monitor a person's health and the impact of a change to a characteristic of their lifestyle based on collecting spectra of their blood. The lifestyle characteristic may be one or more of diet, exercise, alcohol consumption, or use of a tobacco product. The spectra may be collected both prior to and after the person making the change to their lifestyle characteristic. In some embodiments, the near-to-mid IR spectroscope and data processing methods described herein may be used to determine a value for a difference in the collected spectra within one or more specific wavelengths or ranges of wavelength. Based on spectral data obtained from a group of people, these specific wavelengths or ranges of wavelength may be ones found to indicate an impact from a specific change to a lifestyle characteristic, such as an “experiment” that causes a change in diet or a habit, where an experiment may also be referred to as a “program” herein.

In some embodiments, a person's initial blood sample spectra may be processed to identify specific components (sometimes referred to as a “channel” herein) that may be monitored to evaluate the impact of a lifestyle program. Each lifestyle program may be associated with its own set of spectra components or channel that best indicate the impact of the program on blood spectra. Based on the person's initial blood spectra and the channel associated with each program, a process may be used to generate a set of programs that the person could consider trying. This set of possible programs may be filtered based on a demographic characteristic or characteristics of the person, for example by comparing the set of possible programs to a ranked list of programs found helpful by people with a similar demographic characteristic.

In some embodiments, based on a comparison of the person's initial blood sample spectra prior to making a change in a lifestyle characteristic to a reference person (or composite of multiple people) having a similar demographic to the person, the system may generate a recommendation regarding a change to their intake of food, their exercise regimen, or another aspect of their lifestyle that is believed able to improve their health. In some embodiments, the comparison may be between components (or channels) of the person's initial blood sample spectra and those same components of the initial blood sample spectra of a person or persons who then participated in a specific experiment that improved their health.

In some embodiments, the spectral measurements and processing described in the present disclosure do not require identification of a biomarker in a person's blood. Instead the methods described are based on identifying specific components of a person's spectra that are monitored as they undertake a lifestyle change. The specific components may be an intensity of a spectral absorption line or band within specific wavelength ranges that have been found to be correlated with lifestyle changes undertaken by a group of participants. A process for identifying or determining these specific wavelength ranges for each of one or more specific programs or experiments is described in this disclosure.

In some embodiments, a baseline or reference set of these components may be constructed for each of one or more demographic characteristics. This allows a person's initial blood sample spectra to be compared to a standard or reference spectra and used in a process to recommend a specific lifestyle change to cause the person's spectra to become more similar to the standard and/or to be improved in a specific way.

In some embodiments, the system, methods, and apparatus provide an improved user experience that may motivate users to engage in lifestyle experiments to determine the effect of changes in blood spectra related to health, which allows the user to determine which lifestyle changes are likely to improve his or her health. The experiments can be based on measurements of small amounts of blood, and changes in specific components of the blood spectra. The results from these measurements can be tracked with in home spectroscopic measurements, and the change in one or more “channels” or groups of wavelengths or ranges of wavelength reported to the user.

This allows the user to determine which lifestyle activities are likely to improve their health in response to observing changes in these channels. The change in one or more channels or spectra components can be output to a display to allow the user to monitor the change in spectra in response to a change to their lifestyle. By relying on a change in the channel, the approach is less sensitive to the accuracy of the measurements. In some embodiments, a channel is measured at a first time prior to conducting an experiment and at a second time after starting the experiment, and a change or lack of change in the channel is detected. When the experiment has been at least partially completed, the channel readout value is compared to a baseline value prior to initiation of the experiment. The user can conduct a plurality of successive experiments to improve the user's health profile.

In another aspect, the system also comprises a network element communicatively coupled to the spectrometer and configured to process the spectral data to determine a difference between certain components of blood spectra as measured both prior to and either during or after a user participates in an experiment to improve their health, wherein the network element comprises a recommendation engine configured to recommend one or more experiments for the user based on the initial values of the components.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 shows a diagram of an exemplary blood sample and spectrometer, in accordance with some embodiments;

FIGS. 2A and 2B show block diagrams of an exemplary spectrometer measuring spectra of a separating blood sample, in accordance with some embodiments;

FIG. 3 shows a diagram of an exemplary blood sample collector, in accordance with some embodiments;

FIG. 4 shows a block diagram of an exemplary spectrometer with network connectivity, in accordance with some embodiments;

FIG. 5 shows exemplary wavelength plots over time from a spectrometer, in accordance with some embodiments;

FIG. 6 is a flow diagram illustrating a set of data processing operations that may be used to construct a representation of a health component channel, in accordance with some embodiments;

FIG. 7 is a diagram illustrating how the difference in blood sample spectra for a plurality of users participating in an experiment may be used to identify specific spectral components that are responsive to the experiment, in accordance with some embodiments;

FIG. 8 is a flow diagram illustrating a set of data processing operations that may be used to validate a health component channel, in accordance with some embodiments;

FIG. 9 is a diagram illustrating use of a softmax activation function to generate a response from a set of input data, in accordance with some embodiments;

FIG. 10 is a flow diagram illustrating a set of data processing operations that may be used to generate a recommendation for a new user of one or more programs or experiments that may improve their health, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The presently disclosed methods and apparatus will find application in many fields. Although reference is made to testing blood, the presently disclosed methods and apparatus can be used to test many types of biomatrices. The measured biomatrix may comprise a bodily fluid, such as blood, urine, saliva, tears (lacrimal fluid), interstitial fluid, or sweat, for example. In some embodiments, the biomatrix comprises fecal material and the sample comprises a fecal sample. The presently disclosed methods and apparatus are well suited for analyzing any of these samples and obtaining and processing spectral data as described herein.

The presently disclosed methods and apparatus can be incorporated into prior methods and apparatus. For example, although reference is made to a scanning digital mirror, the presently disclosed methods and apparatus can be combined with other types of spectroscopy such as Fourier Transform Infrared (FTIR) spectroscopy, and dispersive spectrometers. For example, the blood collector as disclosed herein can be combined with one or more components of FTIR spectroscopy or dispersive spectroscopy, and combinations thereof. By way of example, the presently disclosed spectrometer may comprise one or more components of the commercially available DLP NIRSCAN Evaluation Module, commercially available from Texas Instruments.

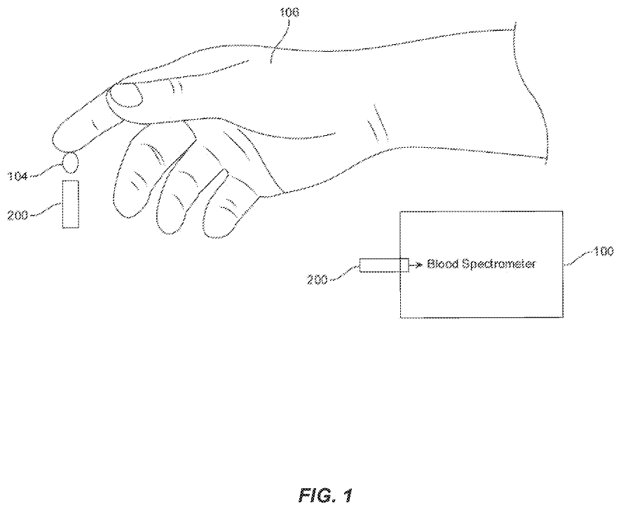

Turning now to FIG. 1, a diagram of an exemplary blood sample 104 and a spectrometer 100 is shown, in accordance with some embodiments. In this embodiment, the spectrometer 100 is configured to receive the sample of blood 104 from a finger of the user 106, although the blood may be sampled from other locations, such as the forearm of the user. In some embodiments, the sample of blood 104 is typically less than a drop of blood which has a volume of about 50 microliters (μL). The sample may comprise a volume within a range from about 0.2 μL to about 5 μL, and the amount of blood can be within a narrower range from about 0.5 μL to about 2 μL, e.g. about 1 μL. The small amount of blood allows the blood sample to be taken from locations that are less painful for the subject. Once a sample of blood 104 is taken from the user 106, the sample holder 200 may be inserted into the spectrometer 100 for spectral analysis. Other bodily fluids can be as described herein sampled similarly with modification of the sampling device. Other materials as described herein can be sampled and measured by the user.

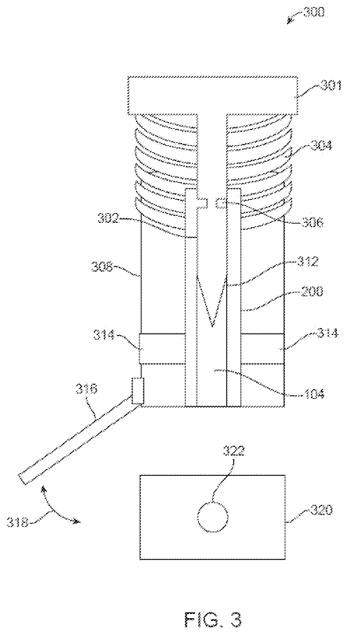

The amount of innervation of the skin can vary depending on the location, and the blood can be drawn at a location of the subject with decreased innervation. For example, the sample can be drawn from the user's forearm 106 with a sample holder, an example of such is shown and described below in FIG. 3. The sample holder may be placed in a receptacle of the spectrometer 100. The sample holder may be configured to allow the blood 104 to at least partially separate into various components. As the blood 104 separates or at least partially separates within the sample holder, the spectrometer 100 may selectively measure the various components of the blood 104, and generate a plurality of wavelength spectrum plots and/or other spectral data that correspond to the separation of the components of the blood 104. For example, the spectrometer 100 and/or some other processing system may generate spectral data of the sample at a plurality of wavelengths and a plurality of times corresponding to at least a partial separation of the sample of blood 104 into a plurality of components of the sample.

The sample blood can be combined with an anti-coagulant or blood thinner to decrease clotting when the blood sample has been placed in the sample holder. This can allow the sample to settle gravimetrically without substantial clotting. For example, the sample holder may comprise an anticoagulant prior to placing the blood in the sample holder. The anticoagulant may comprise one more commercially available anticoagulants, such as heparin. In embodiments where it is desirable to measure a clotting rate, the sample can be measured without or with reduced amounts of anticoagulant in order to allow at least partial clotting of the blood.

The at least partial separation of the blood in the sample holder may occur gravimetrically in response to the earth's gravitational field, and in some embodiments without spinning the sample in a centrifuge. The separation can be related to differences in density of components of the blood. The red blood cells, which contain iron, tend to settle toward the bottom of the sample holder, and the blood plasma, which is less dense than the red blood cells, tends to form near an upper portion of the container. In some embodiments, white blood cells settle in a region between the red blood cells and plasma. The spectrometer and processor can be configured to measure this region. The white blood cells and other cells that contain the cellular DNA, tend to settle in a region between the plasma and the settled red blood cells. This region can be referred to as the “buffy coat.” Infrared spectra from this region of the tube comprising the separated blood can provide information related to DNA changes such as methylation. The spectrometer can be configured to measure at least 3 regions of the blood sample, a first region corresponding to the sample, a second region corresponding to white blood cells and a third region corresponding to red blood cells. The spectrometer can be configured to selectively scan each of these three regions with a plurality of successive measurements at appropriate times as described herein.

To illustrate, the bodily fluid as described herein such as blood may be drawn into the sample holder via a capillary action. As the bodily fluid such as blood is stored in the sample holder, the components of the bodily fluid such as blood 104 may separate into a plurality of components, for example, plasma, red blood cells, white blood cells, etc. The spectrometer 100 may illuminate each of the components by directing light to appropriate locations in the illumination window. With the gravimetric separation as described herein, the ratios of the components of blood at different locations in the sample may change, even though the blood sample may not fully separate. In response to the at least partial separation, each illuminated region of the sample may yield a different wavelength spectrum (e.g., multiple wavelengths of light with varying intensities). The spectrometer 100 may detect these various wavelength spectra (e.g., via an optical detector) and process the spectra for analysis (e.g., relative change of a spectra or component of a spectra, graphical display of the wavelength spectrums over time, medical advice, general healthcare advice, etc.). In this regard, the spectrometer 100 may include a processor and various forms of associated hardware, software, firmware, and combinations thereof.

The spectrometer 100 may focus light on a particular region within the illumination window of the sample holder and produce various wavelength spectra. Alternatively, or in combination, a portion of the sample may be imaged onto a detector through the window of the blood collector. For example, the sample holder may separate the blood 104 into its various components. The spectrometer 100 may illuminate the blood 104 and each of its components, at a particular location within the illumination window, as the blood 104 and its components separate within the sample holder. The detector and the processor of the spectrometer 100 may then generate various wavelength spectra over time, which can be processed accordingly.

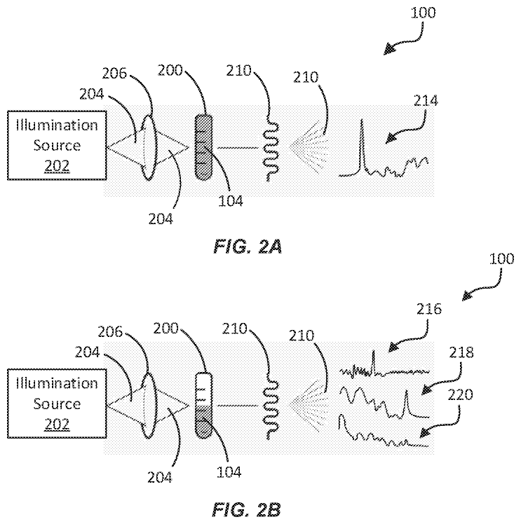

FIGS. 2A and 2B show block diagrams of the exemplary spectrometer 100 measuring spectra (e.g., spectra 214, 216, 218, and 220) of a separating blood sample 104, in accordance with some embodiments. More specifically, FIG. 2A illustrates the spectrometer 100 illuminating the sample blood 104 in a manner that may provide an initial assessment of the blood 104 over a shorter period of time (e.g., within one minute of being drawn). An illumination source 202 of the spectrometer 100 may propagate light 204 through an optical configuration 206 (e.g., various lenses, diffraction grating, mirrors, etc.). The optical configuration 206 may then selectively focus the light 204 to one or more locations of an illumination window of the sample holder 200, which, in turn, produces various wavelengths 210 of light 204. These wavelengths may be detected by an optical detector configured with the spectrometer 100 so as to produce a wavelength spectrum 214.

FIG. 2B shows a similar embodiment where blood 104 has at least partially separated into its various components within the sample holder 200 after a longer period of time (e.g. after at least about 5 minutes of being drawn from the subject). The sample such as a blood sample can be drawn into an elongate transparent structure such as a glass tube, for example. The sample holder 200 may remain in a receptacle of the spectrometer while the blood 104 separates within the holder.

A processor may direct the optical configuration 206 to focus the light 204 at various locations of the illumination window of the sample holder 200 as the blood 104 separates within the sample holder 200. For example, a first ratio of components of the blood may exist at a first location within the sample holder 200, a second ratio of components of the blood 104 may exist at a second location within the sample holder 200, and so on, as the blood separates within the sample holder 200. The processor may direct the optical configuration 206 to focus the light 204 at the various locations to produce different wavelength spectra.

If the blood sample is left undisturbed in the sample holder for a sufficient amount of time, the blood sample may separate into layers corresponding to specific components of the blood sample. In this example, the spectrometer 100 produces an upper wavelength spectrum 216 representative of a user's plasma within the blood 104 at an upper location of the sample holder 200, a wavelength spectrum 218 representative of the user's white blood cells within the blood 104 at an intermediate location within the sample holder 200, and a wavelength spectrum 220 representative of the user's red blood cells within the blood 104 at a lower location within sample holder 200. Although the blood 104 is shown fully separated into different components, work in relation to embodiments of the present disclosure suggests that partial separation of blood is sufficient to provide useful information. Thus, the spectrometer 100 may provide a more in-depth analysis of the user's blood 104.

The illumination source 202 and the optical configuration 206 may alternatively or additionally focus light 204 to a particular location on the illumination window of the sample holder 200. In this embodiment, the sample holder 200 may separate the blood 104 into its various components and propagate those components through the sample holder 200 over time. Thus, the spectrometer 100 may in essence take “snapshots” of the blood 104 and its components over time to generate the wavelength spectra 216, 218, and 220.

One advantage of the spectrometer 100 exists in the placement of the optical configuration 206 between the illumination source 202 and the sample holder 200. For example, by placing the optical configuration 206 between the illumination source 202 and the sample holder 200, the spectrometer 100 may decrease heating of the blood 104 within the sample holder 200. That is, the spectrometer 100 may distance the sample holder from the illumination source 202 in such a way that the blood 104 within the sample holder 200 does not overheat, which can result in measurement errors. In some embodiments, the spectrometer is configured to heat the blood sample by no more than about 5 degrees centigrade when the sample has been placed in the spectrometer and measured for an extended period of time, e.g., for 5 minutes. This limit to heating of no more than 5 degrees C. when placed in the spectrometer for 5 minutes while the blood separates can be helpful for whole blood reagent-less measurements.

Although reference is made to scanning the measured region of the blood sample in FIG. 2B, in some embodiments, the sampled region of blood remains fixed while the blood separates. For example, the light beam can be focused to a location of the sample holder 200 such as an upper location, or collimated light may be passed through a window of the sample holder. The spectra can be recorded as the blood separates at least partially. In some embodiments, the measurement location comprises an upper location of the blood sample. As the blood separates, the red blood cells move away from the upper portion of the column of blood, and the spectra of the upper portion becomes more consistent with spectra of the blood plasma. In some embodiments, measurements of the blood sample from a single location can provide spectral information from the whole blood of the sample and the plasma. Also, the plasma and whole blood information can be used to determine spectral properties of the lower portion of the sample related to a hematocrit of the blood sample based on changes to the spectral signal at the upper location of the blood sample, for example based on subtraction of spectral signals.

Alternatively, or in combination, the measured portion of the blood sample may remain fixed at a lower portion of the blood sample below a midpoint of the blood column. As the blood separates, additional red blood cells are located at the lower portion of the blood column and the sample becomes more consistent with spectra of a hematocrit.

The blood sample can remain placed in the spectrometer for an appropriate amount of time for at least partial separation of the blood to occur, for example gravimetrically. The container may comprise a sealed container to decrease, or even inhibit, drying of the sample (such as a blood sample) during the gravimetric separation. The sample can be allowed to separate for an appropriate amount of time, which can be as short as five minutes, although the separation time may be longer. For example, the amount of time can be within a range from about 5 minutes to about 3 hours, and more specifically from about 30 minutes to 2.5 hours, and for example within a range from about 1 hour to about 2 hours.

The processor can be programmed with instructions for other ranges. For example, the processor can be configured with instructions to spectroscopically measure the sample at a plurality of times within a range from about 5 minutes to about 3 hours while the sample separates, and optionally within a range from about 20 minutes to about 2 hours, and further optionally within a range from about 30 minutes to about 1.5 hours.

A plurality of measurements can be obtained during the time the sample is allowed to separate gravimetrically. The plurality of measurements may comprise successive measurements obtained with an interval of approximately 30 seconds to 10 minutes between measurements, and for example, 1 minute to 5 minutes between successive measurements.

In some embodiments the detector comprises a plurality of detectors as described herein, in which each detector corresponds to a location of the blood sample. For example, a pair of detectors can be used to measure the blood sample at a pair of fixed locations as the blood sample separates, e.g., at an upper location and at a lower location of the blood sample. A grating, digital mirror, interferometer, or other wavelength selector may be scanned or operated to determine the spectra of the sample at the pair of locations.

The spectrometer can be configured in many ways, and may comprise one or more components of known spectrometers, such as a Fourier Transform Infrared (FTIR) spectrometer, a dispersive spectrometer with a detector array, or a spectrometer with a tunable laser as described in U.S. application Ser. No. 14/992,945, filed on Jan. 11, 2016, entitled “Spectroscopic measurements with parallel array detector”, published as US20160123869A1 on May 5, 2016, the entire disclosure of which is incorporated herein by reference. The spectrometer may comprise one or more components and processes as described in PCT/US2019/030052, filed on Apr. 30, 2019, entitled “Systems and methods for blood analysis”, published as WO2019213166, the entire disclosure of which is incorporated herein by reference.

In some embodiments, the spectrometer comprises a tunable laser, for example.

FIG. 3 shows a block diagram of an exemplary blood sample collector 300, in accordance with some embodiments. The blood sample collector 300 comprises a housing 308 to support structures of the blood collector 300. The blood sample collector 300 may comprise a lancet needle 302. The lancet needle 302 may be made from many materials, including but not limited to surgical grade steel. The lancet needle 302 may be sized to extend out of an end of a tube 312 configured within the sample holder 200. The sample holder 200 may be configured within housing 308 of the blood sample collector 300. A user may thus depress the lancet needle 302 through the opening of the sample holder 200 to draw the blood 104 from the user (or another) by pushing a button 301 affixed to an end of the lancet needle 302. In this regard, the blood sample collector 300 may also include a spring mechanism 304 that allows a user to depress the lancet needle 302 into the user's skin to penetrate the user's skin. When the user releases pressure from the button 301, the spring 304 retracts the lancet needle 302 from the user's skin thereby drawing blood 104 into the tube 312 of the sample holder 200 (e.g., via capillary action, suction, or the like).

In some embodiments, the tube 312 comprises a substantially transparent elongate container comprising an elongate axis to separate the sample of blood into the plurality of components. In some embodiments, the tube 312 comprises a capillary tube configured to separate the sample of blood into the plurality of components. In some embodiments, the tube 312 has a volume within a range from about 0.5 to about 2.0 microliter.

The amount of retraction may be limited by an O-ring groove 306 in which an O-ring may be disposed. For example, when the user releases pressure from the button 301 and the lancet needle 302 retracts, the O-ring may limit the amount of retraction to the upper portion of the sample holder 200, thereby retaining the lancet needle 302 within the blood sample collector 300.

When the blood 104 is retained within the sample holder 200 of the blood sample collector 300, the blood sample collector 300 may be closed and/or otherwise sealed with a lid 316. For example, the lid 316 may be attached to the blood sample collector 300 via a hinge mechanism that allows the blood sample collector 300 to open and close as indicated by the angular direction 318. The lid 316 may close the blood sample collector 300 via a compression fit, or other attachment mechanism. However, other embodiments may include attaching the lid 316 to the blood sample collector without a hinge (e.g., via compression fit or other attachment mechanism).

Also illustrated in this embodiment is a guide mechanism 320 that may allow the blood sample collector 300 to accurately draw the blood 104 of the user from a specified location on the user's skin. For example, the guide mechanism 320 may comprise an adhesive that sticks to the user's skin. The guide mechanism 320 may comprise an aperture 322 that is approximately the same size as the tube 312 through which the lancet needle 302 traverses. Thus, when the blood sample collector 300, and more specifically the tube 312, is placed proximate to the user's skin in the aperture 322 of the guide mechanism 320, the lancet needle 302 may penetrate the user's skin through the aperture 322.

The tube 312 may be configured of an optically transparent material, such as glass, plastic or the like. The blood sample collector 300 may be configured with optical ports 314 such that light from an illumination source, such as the illumination source 202 of FIG. 2A or 2B, may pass through the blood sample collector 300 and through the blood 104 to a detector of the spectrometer 100 for subsequent wavelength spectra processing. In this regard, the sample holder 200 may also have optical ports 314 and/or be configured from an optically transparent material capable of propagating light through the blood 104 contained within the tube 312. In some embodiments, the sample holder 200 comprises a slit aperture configured to direct light through the substantially transparent tube 312. The long axis of the slit aperture may be aligned with a long axis of transparent tube 312.

In some embodiments, the sample holder 200 may be configured to provide reagent-less whole blood spectroscopy. In some embodiments, a volume of the sample holder 200 is within a range from about 0.25 microliters to about 4 microliters and optionally within a range from about 0.5 to about 2 microliters. In some embodiments, a height of a window in the sample holder 200 is within a range from about 1 mm to about 20 mm, and optionally within a range from about 2 mm to about 10 mm.

The sample holder 200 may be placed in the spectrometer with or without sample collector 300. In some embodiments, sample collector 300 is placed in the spectrometer with the lancet 302 within the sample holder 200 and the spectra measured. The spectrometer may comprise a receptacle sized and shaped to receive the sample collector 300. The receptacle of the spectrometer may comprise a channel sized and shaped to receive the housing 308 of the sample collector 300. Alternatively, or in combination, the receptacle of the spectrometer may be sized and shaped to receive the sample holder 200 without the housing of the sample collector.

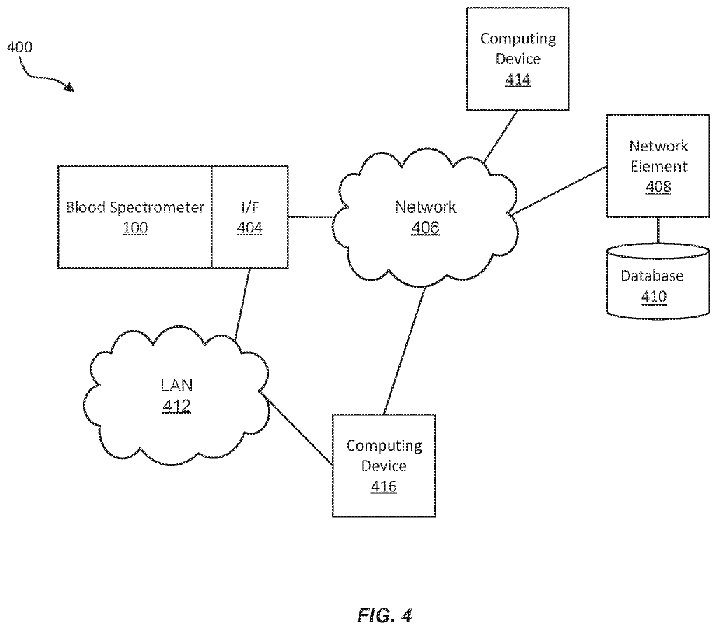

FIG. 4 shows a block diagram of an exemplary spectrometer 100 with network connectivity, in accordance with some embodiments. The spectrometer 100 may be configured with, or coupled to, a network interface 404 that is communicatively coupled to a network 406 (e.g., the Internet) and/or a local area network (LAN) 412. For example, the spectrometer 100 may be configured to receive a sample of blood contained within a sample holder, such as the sample holder 200. The spectrometer 100 may illuminate the sample of blood as the blood at least partially separates within the sample holder. A processor operatively coupled to the spectrometer 100 and/or configured with the spectrometer 100 may be configured with instructions to generate spectral data of the sample at a plurality of wavelengths and a plurality of times corresponding to at least partial separation of the sample of blood into a plurality of components of the sample.

When the spectrometer 100 detects the wavelength spectra of one or more of the components of the blood 104, the spectrometer 100 may communicate the information pertaining to the wavelength spectra and/or the spectral data (e.g., spatially resolved spectral data acquired at a plurality of times) to the network 406, which may in turn communicate the wavelength spectra and/or other spectral data to a network element 408 for subsequent processing. In this regard, the network element 408 may include, or be communicatively coupled to, a database 410 which may comprise various statistics and data pertaining to blood components that can be compared to and/or analyzed against the wavelength spectra of the blood 104. Alternatively, or additionally, the spectrometer 100 may include processing capability that communicates other relevant information pertaining to the blood 104 through the network 406 to the network element 408.

Also illustrated in this embodiment is a computing device 414 that is communicatively coupled to the network 406. The computing device 414 may be used to perform analysis of the wavelength spectra and/or other spectral data of the blood 104 obtained from the spectrometer 100. In this regard, the computing device 414 may be in communication with the network element 408 to retrieve information pertaining to blood analysis such that a user of the computing device 414 (e.g., a medical professional, a trainer, or the like) can analyze the wavelength spectra from the spectrometer 100 and provide a diagnosis and/or other relevant information pertaining to the user's blood 104 to a user of the spectrometer 100. Examples of the computing device 414 include computers, smart phones, and the like, comprising various hardware, software, and/or firmware components for processing the wavelength spectra from the spectrometer 100.

In some embodiments where the spectrometer 100 is communicatively coupled to the LAN 412, the spectrometer 100 may be able to communicate wavelength spectra to a computing device 416. For example, the computing device 416 may also include computers, smart phones, and the like, comprising various hardware, software, and/or firmware components for processing the wavelength spectra and/or other spectral data from the spectrometer 100. In this regard, the computing device 416 may be that of the user using the spectrometer 100. For example, a user may draw his or her own blood 104 using the blood sample collector 300 of FIG. 3. The user may then input the sample of blood 104 into the spectrometer 100 to detect the various wavelength spectra of the components of the blood 104. The spectrometer 100 may then communicate the wavelength spectra to the user's computing device 416 such that the user may process the information and assess the user's own health.

The spectrometer 100, the computing devices 414 and 416, and the network element 408, either alone or in combination, may be configured with instructions (e.g., software components) that direct a processor to perform one or more types of data analyses. For example, a processor configured with the spectrometer 100, the computing devices 414 and 416, and/or the network element 408 may measure a sample of blood and generate spectra comprising absorption features for the sample.

In some embodiments, the processor is configured with instructions to measure the sample at a plurality of times as described herein. The processor can be configured to measure the blood sample at a plurality of times within a range from about one minute to about 3 hours (or longer) while the sample separates. The amount of time the sample is allowed to separate with measurements being obtained can be within a range from about 5 minutes to about 3 hours, more specifically from about 30 minutes to 2.5 hours, and for example, within a range from about 1 hour to about 2 hours. The plurality of measurements may comprise successive measurements obtained with an interval of approximately 30 seconds to 10 minutes between measurements, and for example, 1 minute to 5 minutes between successive measurements.

The processor can be configured with instructions to enable a user to conduct an experiment or participate in a program with a plurality of blood samples from the user (or another subject). For example, the processor can be configured for the user to select one or more experiments (programs) as described herein, such as a specific diet, exercise regimen, or change to an existing habit (such as to stop smoking, reduce alcohol consumption, etc.), or adoption of a new habit (such as meditation).

In response to the user selecting an experiment, the processor provides appropriate prompts for the user to conduct the experiment. The processor may comprise instructions to present an appropriate instruction to the user to conduct the experiment. For each type of experiment, the processor can be configured with instructions to measure a difference in the user's blood sample spectra between an initial sample obtained prior to the experiment and from a sample obtained during or after completion of the experiment. In some embodiments, the processor may determine the difference in spectra and then determine the difference in one or more components of the spectra, referred to as a channel herein.

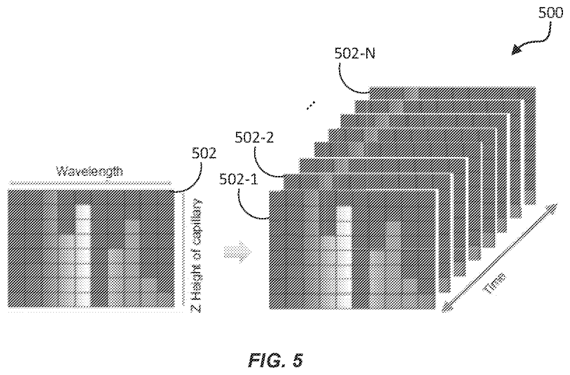

FIG. 5 shows spatially resolved spectral data 500 over time from a spectrometer, in accordance with some embodiments. The spatially resolved spectral data 500 may comprise a plurality of spatially resolved spectral measurements acquired at each of a plurality of times.

An exemplary plot 502 of the spatially resolved spectral data 500 shows the intensity at each of a plurality of wavelengths for each of a plurality of heights of the blood column in the sample holder, such as a capillary tube as described herein. The spectrometer can be configured to measure the spectra of the blood sample at a height Z in the column of blood as the sample separates. The height Z can range from about 1 mm to about 20 mm, for example from about 2 mm to about 10 mm. The number of spatially resolved sample locations along the height Z can range from about 2 to about 1000, for example within a range from about 5 to about 100. The mirror, phase modulator, grating or other wavelength selective component under computer control can be configured to measure the spectrum of the sample at each of the plurality of spatially resolved locations along the height of the sample.

The spectra are recorded for each location along the height of the column. The processor can be configured with instructions to measure each of a plurality of spatially resolved spectra, starting with a first spatially resolved spectral data corresponding to a first plot 502-1 at a first time, followed by a second spatially resolved spectral data corresponding to a second plot 502-2 at a second time, up to Nth spatially resolved spectral data acquired at Nth time and corresponding to an Nth plot 502-N. The spatially resolved spectral data can be measured while the blood sample separates and stored by the processor as described herein.

As disclosed herein, spectral data from a sample of a person's blood may be used to assist them to monitor the impact of a change to their lifestyle on their health. In some embodiments, this may comprise determining a difference between the value(s) of certain components of the spectra between a blood sample obtained prior to undertaking the change in lifestyle and a sample obtained during or after the change. In some embodiments, the components may be termed a “health component channel” herein. The components of the channel are determined based on processing a set of spectral data obtained from blood samples of a group of people who engaged in one or more experiments to determine which aspects of the spectra are responsive to the experiment. In this way, a set of such health component channels may be identified and constructed with each representing those aspects of a blood sample spectra that indicate a response to a particular experiment or set of experiments.

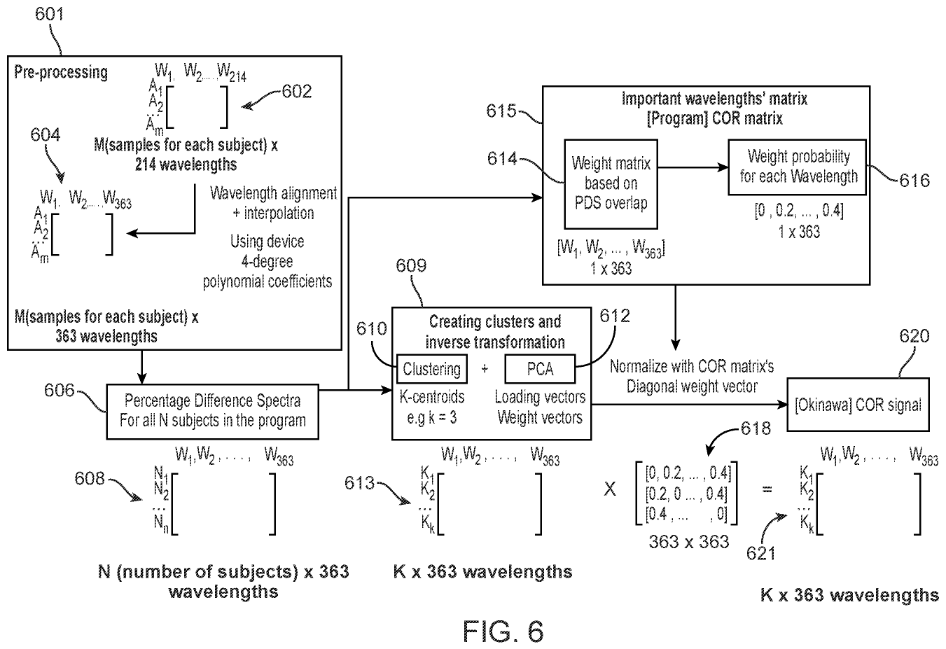

FIG. 6 is a flow diagram illustrating a set of data processing operations that may be used to construct a representation of a health component channel (also referred to as a pure component channel), in accordance with some embodiments. In some embodiments, a group of people are asked to participate in an experiment to determine the impact of the experiment on the spectra of their blood. The group of people may be segmented according to a demographic characteristic or characteristics (such as, but not required to be or limited to age range, gender, ethnicity, race, location, socio-economic grouping, etc.). For each person in the group a sample of their blood is obtained prior to participating in the experiment (or program), where the experiment may comprise a lifestyle change such as a specific diet or exercise regimen, a change to an existing habit (such as to stop smoking or reduce alcohol consumption), or the adoption of a new habit (such as meditation). Each person then participates in the experiment. Participation may involve changing their diet or exercise practices for a period of weeks, for example. During and/or after the experiment, a second sample of blood is taken.

Each of the samples of blood are placed into an infrared spectroscopy system or device, such as the one described herein. The samples may be placed into the device either contemporaneously with when they are collected, or later after being refrigerated or otherwise preserved. Each sample may be illuminated and used to generate a set of absorption lines or bands. Generation of the absorption lines or bands may occur at one or more times or locations of the sample. Generation of the absorption lines or bands may be obtained from whole blood and/or blood plasma depending upon the separation of the sample.

A difference in the spectra between that of the sample obtained after the experiment to that of the sample obtained prior to the experiment is determined. The difference is used to identify those components (that is the absorption lines or bands) of the person's blood that showed a sufficient change and type of change during the experiment. Given a large group of people participating in an experiment, it is possible to identify the components of a person's blood sample spectra that are expected to change as a result of their participation in a specific experiment. Further, based on the demographic segment of the people who participated in the experiment, it is also possible to identify the components of a person's blood sample spectra that are expected to change as a result of their participation in a specific experiment for each of several demographic segments. In addition or instead, it is possible to generate a recommendation to a person of an experiment that those with a similar demographic characteristic have found to be effective in improving their health, as reflected by their blood spectra after participating in the experiment. In contrast to conventional approaches, in the embodiments described herein, this is accomplished without identifying a biomarker in a person's blood sample.

In some embodiments, a process or data processing pipeline for identifying the components of a person's blood sample spectra that are expected to change as a result of their participation in a specific experiment may have the following characteristics and may be implemented as described in the following.

The process correlates nutrition and fitness changes in a cohort to specific absorbance spectrum changes in blood samples over the same time period. As mentioned, no identification or knowledge of blood biomarkers is required. The technology platform described with reference to FIGS. 1-4 is sensitive to components in blood at concentrations as low as 1 mg/dL, and in some cases, lower (as a result, blood changes below this level will not be visible to the spectrometer). Blood absorbance spectrum changes may be related to known or unknown changes in the chemistry of blood—because of this, embodiments do not rely on knowing a specific cause. This is advantageous, as little is now known of day to day and week to week chemistry changes in blood in a healthy population, as laboratory medicine is mainly concerned with disease states.

As shown in FIG. 6, the process may begin by assembling a group of participants willing to perform one or more specific nutrition and fitness programs (as mentioned, these are also referred to as “experiments” herein). The participants should preferably be relatively healthy adults, who are not under the care of a physician for a specific ailment.

Next, the process may include collecting a series of absorbance spectra of blood samples both before and during or after a lifestyle change program using the described blood sample collection device and cartridge. For example, this collecting may include recording the whole blood and plasma spectra and time of sedimentation, where the sample and spectra collection typically involve a few microliters of blood in a glass tube/cartridge, with the tube sealed with gaskets. The tube is held vertically in a spectrometer; the initial scans are of whole blood and the scans transition to blood plasma over time via gravity-based separation.

Each lifestyle change program requires certain fitness and fitness activities (or dietary changes) each day over a multi-day period. As an example, initially, a program may require 7 steps each day over 21 days. If desired, a person may record compliance using a To-Do list in an application on a device.

Next, the process may include pre-processing at 601 by creating a data set of difference spectra. In some embodiments, the first absorbance spectrum at the beginning of a program is subtracted from each subsequent spectrum point-by-point in absorbance space. As an example, each raw absorbance spectrum contains −90 scans in two phases (whole blood and plasma); from each absorbance spectrum the system may obtain a mean whole blood spectrum (the average of the whole blood scans) and a mean plasma spectrum (the average of plasma spectrum). The subtraction is performed in the two phases (whole blood and plasma) and in each phase the subtraction is performed wavelength by wavelength. As an example, in some embodiments, the wavelength range of the spectrometer is roughly 1600-2400 nm (separated by 3.7 nm), so 214 wavelength differences may be computed: W1-W1′, W2-W2′, . . . , Wn-Wn′. Element 602 in FIG. 6 represents a matrix containing m spectra samples (indicated as A1, A2, . . . Am) for each of 214 wavelengths that may be generated for each subject in a program.

In some embodiments, the 214 samples may be processed to align wavelength “bins” for the spectral data and/or to interpolate the acquired data. In some embodiments, the samples may also be fit to a curve and then sampled to generate samples for 363 equally spaced wavelengths, as suggested by element 604. The spectral measurement data in matrix 604 is used as one input to a process to determine a difference in spectral data for each subject by comparing the data in 604 after participation in a program to the data obtained for each subject prior to participation in the program. This processing is described below and represented by element or processing step 606 in FIG. 6.

An example of the spectral difference processing, referred to as the Percentage Difference Spectra (“PDS”) herein, and represented by element or processing step 606, will be further described. The processing may begin by collecting the m absorbance spectra per subject A: {A1, A2, A3, . . . , Am}; thus A1=[a11, a12, a13, . . . , a1n]; A2=[a21, a22, a23, . . . , a2n]; . . . ; Am=[am1, am2, am3, . . . , amn]. The number of spectra, m, per subject may be different per subject.

Next, the process may include computing the difference spectra: {D2, D3, . . . , Dm}, where D1=A1-A1 (all zeros; ignore); D2=A2—A1; D3=A3—A1; . . . ; Dm=Am-A1. For each difference spectrum, the process may include determining whether the value for each value is above zero or not: {T2, T3, . . . , Tm}; thus T2=[t21, t22, t23, . . . , t2n]; T3=[t31, t32, t33, . . . , t3n]; Tm=[tm1, tm2, tm3, . . . tmm], where each value of tij is 0 or 1 (1 if the value tij>0; 0 otherwise).

Next, the process may include computing the PDS spectrum, where PDS=[p1, p2, p3, . . . , pm], and each value of pi is an average of the corresponding t values, i.e., pi=(t2+t2+ . . . +tm)/(m−1)*100. A PDS spectrum may be computed for each subject; a large value of pi indicates that the ith wavelength is more important study-wise (a more meaningful indicator of change) than if the value of pi is lower. For purposes of display and visualization, in some examples, each wavelength may be associated with a color, such as blue if pi>60% and orange otherwise (or for other values, such as 70%).

In some embodiments, an output of the above described PDS processing is a matrix of the form of element 608, containing, for each subject Ni in a specific program, a difference in their spectra after participation in the experiment for each of a set of wavelengths, Wi (as mentioned, in one example 363 wavelengths).

Once the percentage difference spectra are computed for each participant, the components of the spectra that are impacted by the experiment may be determined. In some embodiments, this is termed a “health component channel” (although as mentioned, this may also be referred to as a principal component channel or pure component channel). This may be accomplished at step 609 (creating clusters and inverse transformation) by use of a data processing algorithm such as principal components analysis (PCA) in combination with a clustering methodology. Clustering and PCA may be used to select a set of wavelengths and absorbances that can be extracted from the set of difference spectra and that have the following desirable characteristics, as suggested by element or data processing step(s) 610 and 612.

Firstly, it is desirable to choose wavelengths which exhibit a unidirectional change in absorbance from the beginning to the end of the experiment. A threshold for finding wavelengths that exhibit such a unidirectional change across all differenced spectra will typically be dynamic, but >60%; on a wavelength-wavelength basis is a percentage of unidirectionality exhibited that may be used. Wavelengths that exhibit little of any change across all subjects are assumed to be less informative, while wavelengths that exhibit the most unidirectionality across subjects are assumed to be the most informative. Further, a feature importance score may be computed. This may be based on using a filtering method that evaluates the importance of features (wavelengths) based on their inherent characteristics (e.g., multi-cluster feature selection (MCFS), variance, dispersion ratio, etc.).

An example algorithm or data processing method that may be used to identify a set of wavelengths believed to have a desirable behavior in response to the experiment by implementing a version of multi-cluster feature selection may be similar to the example pseudocode contained in an article entitled “Unsupervised Feature Selection for Multi-Cluster Data,” Deng Cai, Chiyuan Zhang, and Xiaofei He, State Key Lab of CAD&CG, College of Computer Science, Zhejiang University, China, KDD '10: Proceedings of the 16th ACM SIGKDD International Conference on Knowledge Discovery and Data Mining, July 2010 Pages 333-342.

The example data processing method may include constructing a p (number of nearest neighbors) nearest neighbor graph, and solving a generalized eigen-problem (e.g., Y=[y₁, . . . , y_{K}] for the top K (number of clusters) eigenvectors with respect to the smallest eigenvalues). The method may also include solving K L1-regularized regression problems with the cardinality constraint set to d (number of selected features) to get K sparse coefficient vectors. The method may further include computing the MCFS score for each feature and returning the top d features according to their MCFS scores.

Secondly, it is desirable to select wavelengths which exhibit this unidirectional change across a significant enough number of the participants. As an example, in each 21-day program, there are 7 steps, sometimes referred to the “Daily To-Dos”; at the moment at-least 1 or 2 of the 7 To-Dos are validated interventions known to have an effect on blood chemistry. For example, increased intake of fish is validated to an increase in Omega 3 levels in the blood. Therefore, in one of the experiments or programs, participants are asked to eat fish every day for 21 days. One way to estimate the number of participants needed to be “sufficient” is using existing work on the validated daily To-Do. Using this information, one can estimate the number of participants needed to see a significant change in the same To-Do item based on the expected effect size and the reported mean and standard deviation of the study.

In some embodiments, in order to have a preliminary confidence that the change in blood chemistry is significant, the change is expected to be observed in more than 60% of the participants that undergo the lifestyle change or experiment (although the number of participants that undergo the lifestyle change/experiment may not be the same as the total number of participants across all experiments). Using a clustering technique, one can identify homogeneous principal components and patterns of change in a significant number of participants. After removing the water-band (the absorption features arising from water molecules), one may consider two Regions (regions 1 and 2). Region 1 is region associated with the shorter wavelengths to the left of the water-band (in wavelength space, as expressed in nm) and region 2 is the region associated with the longer wavelengths to the right of the water band. Historically, most of the wavelength change has occurred in the longer wavelength region (i.e., region 2). Hence, more significance may be given (if desired) to region 2 than to region 1.

With respect to “significance,” one can define a percentage threshold, such as 60%. For a given subject, one then finds the number of wavelengths that meet this threshold. This defines a “differentially interesting” subset for this person or subject. One can do this evaluation for all subjects, and see which wavelengths maximally overlap in their differentially interesting subsets. In some cases, there may be timepoints where a subject has all of its interesting wavelengths change unidirectionally. These timepoints could be deemed interesting “temporal” landmarks as well and contribute to tuning the significant threshold values. The information regarding wavelength overlap can be used to generate a weighting matrix to be applied to the components (the percentage difference values for each wavelength) of significance found from the clustering 610 and PCA 612 processing. In FIG. 6, this weighting matrix is represented by element 614. The weighting matrix based on overlap 614 may be normalized and converted to a weight probability matrix having a value between 0 and 1 for each of the 363 wavelengths, as suggested by element 616. In some embodiments, the processing also constructs a “COR matrix” at 615 for the program/experiment, which represents the weight of important wavelengths seen in a significant number of participants, in the form of a probability matrix of dimension 1×363 (as suggested by elements 614 and 616).

Returning to the clustering 610 and PCA 612 processing at step 609, the output of the two data processing steps is a matrix 613 whose elements represent a cluster assignment K defined in a K-means clustering algorithm and its feature mean for each wavelength Wi, when a number k of centroids is used in the clustering algorithm. This assists in determining the “best” number of clusters to use for the further analysis. More specifically, K1, . . . Kk are the clusters defined after the clustering analysis of the data. For each of the K clusters (K1, . . . Kk), a cluster centroid is computed. The kth cluster centroid is the vector of the feature means for the observations in the kth cluster.

In some embodiments, to identify various wavelengths of interest in the blood spectra data, the processing implements a K-means clustering algorithm and PCA. The processing recovers specific data points from the clusters and evaluates their significance by transforming them back into their original dimension and scale. Based on a silhouette coefficient, the process selects the optimal number of clusters for each model. In some embodiments, the processing also constructs a “COR matrix” for the program/experiment at step 615, which represents the weight of important wavelengths seen in a significant number of participants, in the form of a probability matrix of dimension 1×363 (as suggested by elements 614 and 616).

Matrix 613 which resulted from the clustering and PCA processing may then be multiplied by a matrix 618 constructed from the weighting probability vector 616 to form a normalized and weighted version of the significant features of the spectra represented by matrix 613 that represent a response to the experiment. This may be termed a Program or Experiment's COR signal 620, which may be in the form of a matrix 621. In the figure, the example shown is identified as [Okinawa] COR Signal, indicating that it represents the blood spectra elements observed to change sufficiently for a sufficient number of subjects to be considered relevant when those subjects participated in practicing the Okinawa diet.

One may define this pure component spectrum or COR Signal as a “channel” for further analysis and tracking. The spectral pattern of the channel is a result of the chemical change (as indicated by the absorption spectra) in a subject's blood that is believed to be correlated with the nutritional and/or fitness change or changes that are part of the experiment.

In some embodiments, a pattern may be assigned/designated based on two metrics: explained variance and factor loading. Explained variance is how much the pattern can reflect the variance of the whole change, and factor loading is how much a variable correlates with a component. In some embodiments, each channel may be constructed from a linear combination of wavelengths, where some wavelengths may have more weight than others. As described, in some embodiments, the wavelengths that have the most overlap with respect to their differentially interesting subsets are deemed the pattern, i.e., the wavelength “assay.” Note that there may be a large number of possible patterns that can be derived from a set of spectral data.

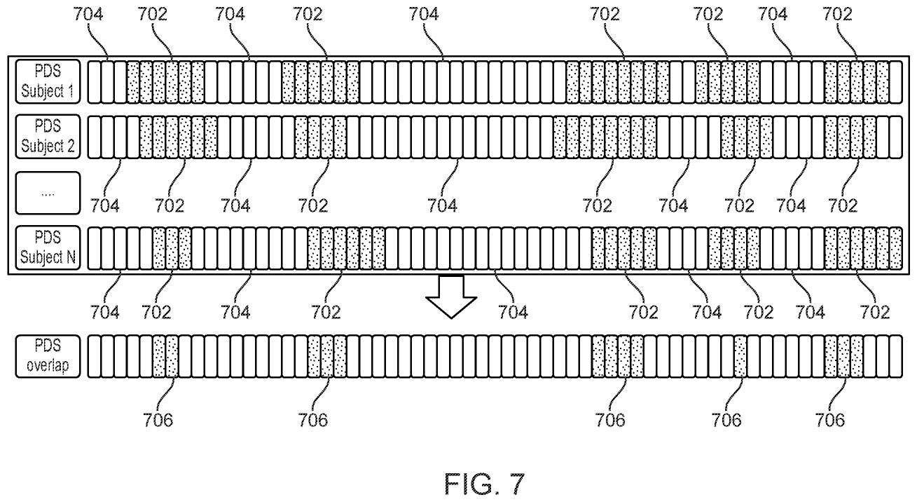

FIG. 7 is a diagram illustrating how the difference in blood sample spectra for a plurality of users participating in an experiment may be used to identify specific spectral components that are responsive to the experiment. As shown in the figure, the percentage difference spectra for a set of N subjects (indicated as “PDS Subject 1”, . . . , “PDS Subject N” in the figure) may be used to determine the overlapping or most likely to be informative wavelengths with regards to the impact of a specific experiment on the subjects' blood chemistry. In the figure, the most likely to be significant wavelengths are indicated for each subject by the wavelengths with element number 702, while the less likely to be informative wavelengths for each subject are indicated by the element number 704. The wavelengths labeled as “PDS Overlap” represent the most likely to be informative wavelengths that are common to or are shared by the subjects and are indicated by element 706.

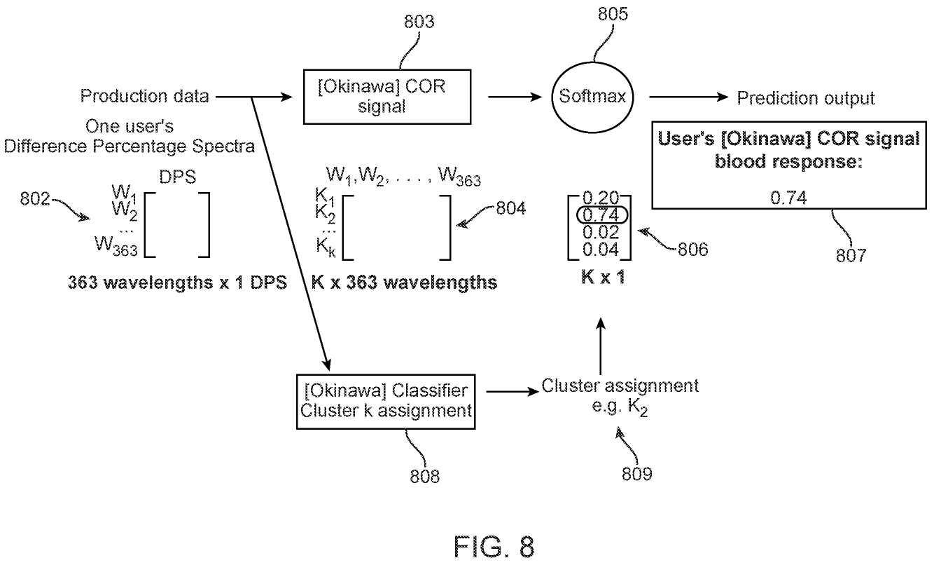

Given a COR Signal, channel, or model representing a set of wavelengths that are believed to be most indicative of blood chemistry changes that are correlated with participation in a specific program or experiment, a validation process may be implemented to evaluate the accuracy and reliability of the COR signal. FIG. 8 is a flow diagram illustrating a set of data processing operations that may be used to validate a health component channel, in accordance with some embodiments.

As shown in the figure, difference percentage spectral data for a subject whose data was not used in developing the COR signal or channel (sometimes referred to as hold-out data) may be used as an input to the processing flow, as suggested by element 802. This data matrix may be used with the COR signal for a specific experiment, as indicated by [Okinawa] COR signal matrix 804 in the figure. A normalized exponential algorithm (e.g., a softmax activation algorithm) or processing step is then used to evaluate the accuracy and reliability of the processes used to generate the COR signal by generating an output that represents the cluster assignment of the data in matrix 802 and a numerical value representing the blood chemistry response of the subject on a scale from 0 to 1, as suggested by data element 806.

FIG. 8 represents the data flow both for a validation process and also a process that may be used with a new user. Once a user has more than 1 blood sample, a difference probability spectra (DPS) can be calculated, as in element 802. This data can then be multiplied by a COR signal 803 (e.g., an [Okinawa] COR signal matrix) to produce a version of the matrix 804 for the new user. In some embodiments, the COR signal matrix corresponds to change from an experiment such as a diet, e.g. the Okinawa diet. The COR signal result can further be subject to a softmax operation 805 and represented as a percentage for each k cluster (which may be assigned based on a classifier cluster assignment 808 and cluster assignment 809), as suggested by data element 806. The percentage of the cluster deemed closest to the user's matrix 802 is then selected as the user's blood response 807 to the COR signal. To classify the cluster (e.g., at steps 808 and 809) a classification model based on clustering process 610 is used that outputs the cluster whose centroid is closest to 802, where closest is defined using a Euclidean distance metric.

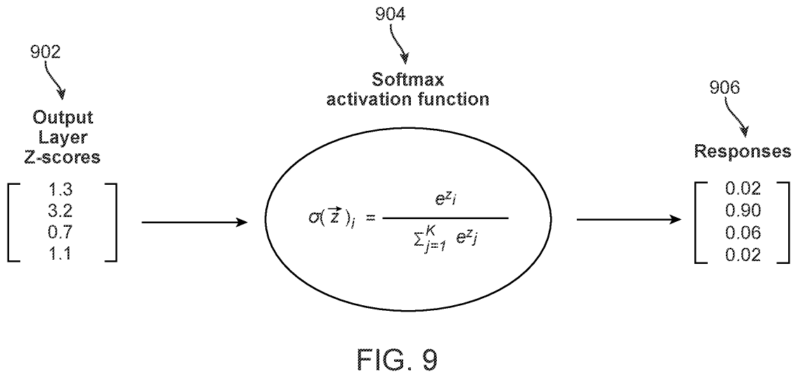

FIG. 9 is a diagram illustrating use of a softmax activation function 904 to generate a response 906 from a set of input data 902, in accordance with some embodiments.

One or more COR signals or channels may be generated, with each representing a set of wavelengths believed to show changes in blood chemistry as the result of a subject participating in a specific experiment. In some embodiments, the set of COR signals may be used as part of a process to generate a recommendation for a new user regarding an experiment or experiments that they should consider participating in to improve their health.

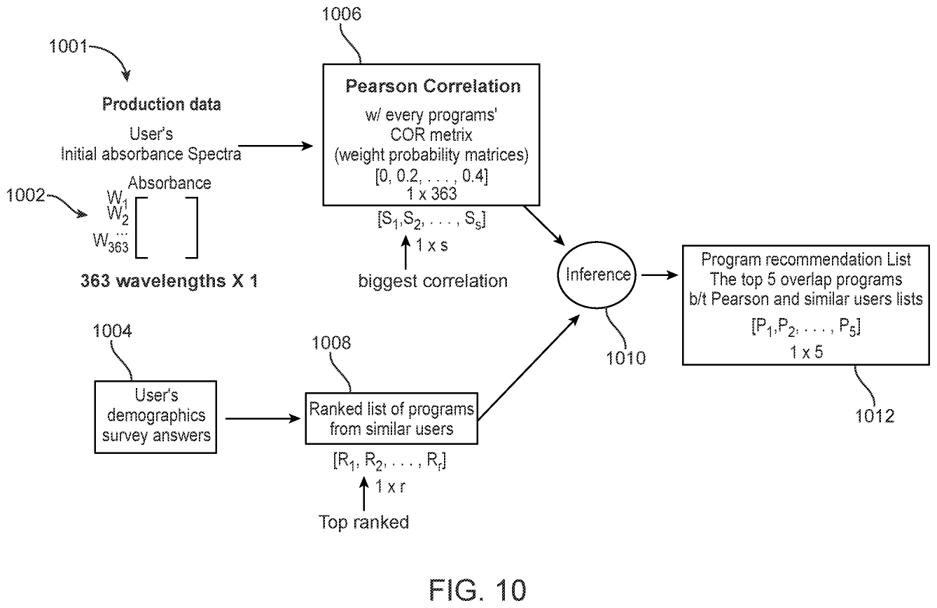

FIG. 10 is a flow diagram illustrating a set of data processing operations that may be used to generate a recommendation for a new user of one or more programs or experiments that may improve their health, in accordance with some embodiments. As shown in the figure, the process may start at step 1001 with collection of a new user's absorption spectra from a blood sample of the new user that is obtained prior to the new user participating in a program or experiment. This spectral data may be represented in the form of an absorbance value for each of a set of wavelengths, as suggested by matrix element 1002. A second input to the process flow may be the new user's answers to a set of demographic survey questions, as suggested by element 1004.

The process flow then determines a correlation between the new user's initial absorption spectra data 1002 and each program's COR signal matrix (the result of the processes described with reference to elements 614 and 616 of FIG. 6), as suggested by Pearson Correlation process element 1006. The Pearson correlation coefficient (PCC) is a measure of the linear correlation between two sets of data and is defined as the covariance of two variables, divided by the product of their standard deviations; it is essentially a normalized measurement of the covariance, such that the result has a value between −1 and 1. The output of the correlation process is a vector representing the degree of correlation between the new user's absorption spectra and each of the “important” or “informative” wavelengths identified for an experiment. This provides an indication of a set of experiments for the new user that may be best able to be monitored by virtue of the new user's blood chemistry.

In some embodiments, a comparison of a new user's initial blood spectra to each program's COR signal may produce a Z-score, or similar measure of how similar or different the new user's spectra is to the program's signal. This may suggest that the new user participate in an experiment or program for which the Z-score indicates a large difference.

The user's demographic survey information 1004 is used to access or generate a ranked list of programs or experiments participated in by those with similar demographic characteristics, as suggested by element 1008. The ranking may be based on a measure of the amount of “success” each similar user had when they participated in an experiment, the number of people with a similar demographic characteristic who participated in an experiment, a measure of the deviation of the new user's blood spectra from the initial or final blood spectra of the participants in an experiment (such as a Z-score or other measure that could be used to determine how far away the new user is from the initial or final state of the participants in a specific experiment), how effective an experiment was at improving the health of people having a similar demographic characteristic, etc. A final ranking may then be constructed from an ensemble (two or more) of the methods mentioned with a linear combination of the results producing the most suggested program or experiment (the one or ones the highest value).

The correlation information output from process 1006 and the ranked list output from process 1008 may be input to an inference process 1010 to generate a list or other form of representing the overlap between the blood chemistry correlation data of the new user and the programs participated in by those with similar demographic characteristics 1012. This may be provided as an ordered list (shown as P1, P2, . . . P5) identifying the top-5 (in this example) programs or experiments recommended to the new user. In some sense, the output 1012 of the inference process 1010 represents those experiments or programs for which the new user's blood chemistry is expected to be able to be used to monitor their health improvement and which were participated in by others of a similar demographic segment. In some embodiments, the inference process or function 1010 operates to ingest data 1006 and 1008 and output a sorted list. In some embodiments, inference function 1010 will generate a score for each element in both lists; the f score is given to the first elements, f−1 to the second element, f−2 to the third and so on. Next, the function merges the two lists and sums the scores of the similar elements. The element with the highest 5 scores may then be listed as the five suggested programs.

In some embodiments, Z-scores or other metrics for one or more channels may be combined to produce a new score reflective of a specific goal. For example, scores for channels that are similar or have similar goals can be grouped together to produce a new score for evaluating a user. As an example, a Longevity Score may be associated with a score for a program in which people are following an Okinawa lifestyle and a program in which people are following an Ikaria lifestyle program, as both of these lifestyle programs are commonly understood in the lifestyle community to be favorable for longevity.

The data processing and workflow can be configured in many ways to define channels and to evaluate the impact of experiments on a user's blood spectral components and to make recommendations. In some embodiments, the expression of a channel in a specific user can be determined using a standard deviation Z-score. The Z-score is the number of standard deviations a particular blood sample is from the mean of a calibration or reference set (or in some cases a channel representing a group of people). A large positive Z-score means that the amount of that pure component (or channel) is large in that sample and a large negative Z-score means the opposite.

In some embodiments, the principal components or channel may be considered a blood response profile (BRP) for a specific person. In this sense it may be viewed as the response to a driving function, in this case a specific experiment or program. In some embodiments, an expression of how responsive a program was for a user may be indicated by a number on a numerical scale of [0,1].

Using a reference or baseline channel, and the user's Z-score in its different components (wavelengths or principal component(s)), along with the mean and standard deviation of program's participants, the blood response profile (BRP) may be calculated to report the highest response found in a component (a principal component or combination of wavelengths) of the program's reference channel. In some embodiments, Z-scores may be input to a softmax activation function, and the highest Z-score reported as the response. Comparing the user in different components of the system's reference against the bigger cohort with the Z-score and applying the softmax function to get each component/Z-score in the interval (0,1), the component with the biggest response will be highlighted. This approach may enable a user to identify and compare different experiments that they've participated in based on the biggest change.

In some embodiments, a baseline or reference channel may be used in a process that generates a recommendation of one or more experiments for a new user to participate in to improve their health. For example, a blood sample of a new user may be processed to identify the same components as in a reference or baseline channel. The blood sample may be obtained prior to the new user participating in an experiment or may be obtained after the new user has participated in an experiment.

The reference or baseline channel may be selected because it represents a person having a demographic characteristic of the new user (such as age, age range, gender, race, etc.). The reference or baseline channel may be formed from spectra obtained from a group of people prior to their participating in an experiment. The reference or baseline channel may be formed from a difference in spectra obtained from a group of people after they participated in an experiment. The reference or baseline channel may be formed from a weighted or other combination (scaled, raised to a power, etc.) of spectra obtained from a group of people who participated in one experiment or in a plurality of experiments. The components of the spectra used to form a baseline or reference channel may be those whose changes are found to be correlated with participation in an experiment.

In some embodiments, a new user's blood spectra may be processed to determine the principal components (e.g., a specific set of wavelengths or wavelength bands) found in a specific health component channel. A reference or baseline channel for comparison to the new user's spectra components may then be selected based on a demographic characteristic of the new user. The difference between the new user's principal components and the reference channel may be used to determine an experiment or experiments to recommend to the new user in an effort to cause their spectra's components to become more like those of the reference. This may be done by determining which experiments are likely to result in a change in the new user's spectra to make its principal components more similar to the values of the reference channel's principal components.

In another embodiment, the principal components for each of a group of people are compared to those of a new user. This may be done for people who participated in one experiment or in more than one. In some embodiments, the principal components of some or all of the group of people may be based on blood samples prior to the people participating in an experiment. Using a nearest neighbor or other form of clustering technique the new user's principal components may be compared to the group of people to identify the person or people that the new user's initial blood sample is closest to, or to identify the person or people that the new user's later blood sample is closest to. In some cases, collaborative filtering may be used to suggest that the new user participate in an experiment that proved successful for a person having a similar starting or after experiment spectra. Alternatively or in combination, a difference in spectra can be calculated between the new user and similar user or users, e.g. with a k-nearest neighbor approach, and a Z-score generated for each of the channels, in which each of the channels is associated with a specific experimental program. If the Z-score from the channel for a specific experimental program indicates a strong correlation for the new user as compared with the Z-score of other experimental programs, that experimental program with the strongest correlation can be recommended to the user to promote changes to the new user's blood spectra that are associated with health. One of ordinary skill in art will recognize that the difference spectrum can be generated in many ways, and in some embodiments, a strong anti-correlation can be used to recommend the experimental program to the new user.

Regardless of the methodology used to generate a recommendation of an experiment or experiments for a new user, they are asked to select and then engage in an experiment. During or after completion of the experiment, a new blood sample is obtained. The difference in spectra between the initial (pre-experiment) and later (during or post-experiment) spectra is determined, using the processing flow described herein. In some embodiments, the principal components of the difference are identified and then compared to the similar components of another person or persons having the same demographic characteristic as the new user and who participated in the same experiment. This allows a direct comparison between the progress or improvement made by the new user and others who are similarly situated. In some embodiments, the difference spectra of the new user may be compared to the COR signal for those who participated in a specific experiment to determine how much closer the new user's blood chemistry has become to those who participated in the experiment. This may provide guidance on how successful the new user has been at improving their blood chemistry and health.

In some embodiments, a combination of one or more health component channels (COR signals) may be used to form a reference or baseline channel for use in comparison to a new user. The baseline channel may comprise a set of spectral lines or bands obtained from mid-IR (e.g., 1600-2400 nm) absorption by a sample of blood. The line or band intensities or other characteristic of the baseline channel may be derived from a set of people in a demographic segment, from a set of people who participated in a specific experiment, or both. The baseline channel may be derived from blood spectra measurements of one or more people prior to the people participating in a specific experiment. In some embodiments, the baseline channel may be used to generate one or more recommendations of experiments for a new user based on comparing the new user's initial spectral components to the baseline channel. In some embodiments, the comparison may be between certain components of a user's initial blood sample spectra and a baseline channel of a person (or persons) in a similar demographic segment as the user.

As described, blood sample spectra from a group of people may be collected, processed, and used to perform one or more of the following: (a) identify those IR absorption lines or bands of the spectra that change in response to a person participating in a specific experiment, (b) construct one or more health component channels representing a set of IR spectral absorption lines or bands that are expected to change when a person participates in one or more experiments, (c) associate each health component channel with a demographic segment (such as based on age range, gender, or ethnicity), and (d) construct a baseline or reference channel for a specific user based on the user's demographic segment. The baseline or reference channel may then be used as part of a process to generate a recommendation to the user regarding which experiment or experiments they should consider trying in order to improve their health.

In some embodiments, a COR program recommendation engine consists of two primary elements, as suggested by FIG. 10: (1) each programs' COR matrix (e.g. [Okinawa COR matrix) or weight probability matrix of wavelengths; and (2) a ranked list of programs from similar users, e.g., users within the same age group and similar activity level. A user's initial blood absorbance spectra is used to generate a Pearson correlation score with each COR program matrix. The user's demographic characteristics (e.g., sex, age group) and/or survey answers (e.g., lifestyle, activity level, diet) are used to access or generate a ranked list of programs from similar users. The top (n) overlapping programs between the two parts/lists may be used to form a list of recommended experiments for the new user. A similar approach can be used to generate a list of recommended To-Dos (activity/diet). In this example, the two parts would be: (1) a COR matrix for each To-Do item, weight probability matrix or other form of representing the important wavelengths for the item; and (2) a ranked list of To-Do items from similar users.

From one perspective, a COR signal is a Composite Infrared Spectroscopic Correlate of Nutrition and Fitness. The Infrared (IR) spectra of whole blood and serum obtained under quantitative conditions contain additional information and can serve as a useful correlate of health. Further, infrared spectra have an advantage of being easy to obtain compared to traditional methods of assaying blood.

The COR signal originates from the molecular structure of proteins, carbohydrates, esters, and lipids. Unlike a lab quant assay, the COR Signal also includes information about molecular secondary structure, chemical environment, and interactions. The amplitude of the COR signal is believed to correlate directly with healthy nutrition and fitness changes, providing a unique and convenient measure for personalized nutrition and fitness fine tuning.

Further, the COR signal can be used to build a normative compendium of data in a normal population that better reflects nutrition and fitness than through the interpretation of disease biomarkers. As described herein, various clustering and analysis techniques may be used to quantify COR signal amplitudes from spectrometer test spectra and demonstrate a correlation with healthy lifestyle programs in a healthy population. The COR signal correlates with a common understanding of healthy lifestyle, wellness, fitness, and nutrition, and has analytic, ease-of-use and accessibility attributes that may complement or provide advantages over existing clinical markers for nutrition and fitness.

Embodiments of the disclosure have been described with reference to generating a recommendation of a program or experiment for a person to participate in to improve their health. However, the system, apparatuses, and methods described may also be used as part of generating motivational assistance for a user and to generate additional metrics for use in comparing a user to other users. As examples, the blood sample data and processing techniques described herein may be used in the following ways: (1) A recommendation algorithm to suggest a COR program that might be relatively effective for that user, because of a phenotypic similarity between that user and others in a cohort; (2) A motivational tool that awards “positive contribution points” or bonus points for successfully completing a COR program; (3) Identifying changes that are determined by a “correlation engine.” For example, once a BRP (blood response pattern) is determined that is correlated to a particular program or nutrition and fitness experiment, that BRP has an associated magnitude and a direction. It improves when certain users do the program, inducing a BRP change; this implies a directionality. To determine a magnitude, the system can do one of the following: (a) Total scale of the change across the population can be scaled as standard deviations away from the mean BRP magnitude; (b) The reference interval can be defined as the 95% of the range; (c) The change can be quantitatively expressed in terms of numbers of standard deviations away from the mean—a +1 for a particular BRP for a particular blood sample would mean 1 standard deviation better than the mean BRP magnitude (this is a traditional Z-score scaling). Additionally, (4) The BRP magnitudes can be plotted as a tracked trend line to inform the user how their recent lifestyle practices are affecting that particular BRP magnitude; and (5) BRP may be organized into “channels”, providing up to date feedback. These BRP channels can be named for the experiment that they correspond to. For example, an Okinawa nutrition, fitness and lifestyle program is created because people in Okinawa experience notable longevity. The program evokes a significant pattern change, a BRP, in a significant number of participants. COR then defines this BRP as a channel, and names the channel “Longevity.” Future blood samples can then report a Longevity Z-score, even outside of an explicit Okinawa experiment.

In this manner, the validated COR Longevity Z-Score may be used on its own as a composite marker of nutritional fitness, lifestyle betterment, and specifically Longevity.

As detailed above, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions, including networked processors such as a server farm. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

The term “network element,” as used herein, generally represents any devices, systems, software, processor, or combinations thereof capable of providing communication through a network. Examples of such include network servers, computing devices, interfaces, databases, storage devices, communication interfaces, and the like.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. For example, one or more of the devices recited herein may receive image data of a sample to be transformed, transform the image data, output a result of the transformation to determine a 3D process, use the result of the transformation to perform the 3D process, and store the result of the transformation to produce an output image of the sample. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

As described herein, the terms “experiment” and “program” are used interchangeably.

This disclosure also includes the following numbered clauses:

Clause 1. A method of recommending a lifestyle experiment to a person, the method comprising: obtaining spectral data from a sample of the person's biomatrix; obtaining demographic characteristics for the person; processing the spectral data to generate correlations for a set of health component channels associated with a set of lifestyle experiments; generating a list of experiments from the set of lifestyle experiments for users with similar demographic characteristics; comparing the correlations for the set of health component channels to the list of experiments to determine overlap between the correlations and the list; and based on the comparison, selecting the lifestyle experiment from the set of lifestyle experiments as a recommended lifestyle experiment for the person.

Clause 2. The method of clause 1, wherein a health component channel of the set of health component channels is identified without identifying a biomarker in the biomatrix sample.

Clause 3. The method of clause 1 or 2, wherein the lifestyle experiment is selected from the set of lifestyle experiments based on a correlation between a health component channel of the experiment and the spectral data from the sample of the person's biomatrix.

Clause 4. The method of clause 3, wherein a plurality of lifestyle experiments is selected from the set of lifestyle experiments based on correlations between corresponding health component channels of each of the plurality of lifestyle experiments and the sample of the person's biomatrix.

Clause 5. The method of clause 4, wherein the plurality of lifestyle experiments is presented to the person for the person to select a lifestyle experiment from the plurality of lifestyle experiments.

Clause 6. The method of any of clauses 1-5, wherein each member of the set of health component channels is associated with an experiment.

Clause 7. The method of clause 6, wherein the set of health component channels is defined at least in part by a weight probability matrix, the weight probability matrix comprising a plurality of weights to be combined with a plurality of values of the spectral data, each of the plurality of values of the spectral data associated with a wavelength of the spectral data.

Clause 8. The method of clause 7, wherein the spectral data of the person comprises a vector of spectral values and wherein combining the weight probability matrix with the vector of spectral values generates a correlation vector, the correlation vector comprising a set of correlations associated with the set of experiments.

Clause 9. The method of clause 8, wherein the lifestyle experiment is selected from the set of lifestyle experiments based on a corresponding value in the correlation vector.

Clause 10. The method of any of clauses 1-9, wherein the demographic characteristics comprise one or more of age range, gender, race, location, highest education level, socioeconomic class, or nationality.

Clause 11. The method of any of clauses 1-10, wherein the demographic characteristics for the person are used to generate a ranked list of experiments from users having similar demographics.

Clause 12. The method of clause 11, wherein a ranked experiment recommendation list is generated from an overlap between the ranked list of experiments and a correlation vector, the correlation vector comprising correlations between corresponding health component channels of each of the set of lifestyle experiments and the sample of the person's biomatrix.

Clause 13. The method of clause 11 or 12, wherein the ranked list of experiments is generated based on one or more of a measure of an amount of success each similar user had when they participated in an experiment, a number of people with a similar demographic characteristic who participated in an experiment, a measure of a deviation of a new user's biomatrix spectra from an initial or final biomatrix spectra of participants in an experiment, or how effective an experiment was at improving health of people having a similar demographic characteristic.

Clause 14. The method of any of clauses 1-13, wherein the lifestyle experiment corresponds to a change a lifestyle characteristic comprising one or more of diet, exercise, drug consumption, alcohol consumption, or use of nicotine.

Clause 15. The method of any of clauses 1-14, wherein each member of the set of health component channels comprises a weighting vector configured to combine spectral data from each of a plurality of wavelengths.

Clause 16. The method of clause 15, wherein said each member of the set of health component channels has been associated with demographic characteristics of prior users to generate the list of experiments based on demographic similarity between the prior users and the person.

Clause 17. The method of any of clauses 1-16, wherein the spectral data is obtained by an infrared spectrometry system measuring a plurality of wavelengths of the sample of the person's biomatrix within a range from about 1000 nm to about 2000 nm, and wherein the spectral data from the sample of the person's biomatrix comprises from about 100 to about 500 intensity values for the plurality of wavelengths.

Clause 18. The method of clause 17, wherein the intensity values for the plurality of wavelengths are combined in accordance with a weighting function to determine an intensity value of an identified health component channel.

Clause 19. The method of any of clauses 1-18, wherein a health component channel comprises a weighted combination of a plurality of values of the spectra of the biomatrix sample at a plurality of specific wavelengths.

Clause 20. A method of generating a dataset to process spectrometer data from biomatrix samples, the method comprising: defining a set of lifestyle experiments associated with a change to a lifestyle characteristic; selecting a set of participants to engage in the set of lifestyle experiments; acquiring first spectral data from a first biomatrix sample for each of the set of participants at a first time to establish a baseline; acquiring second spectral data from a second biomatrix sample at a second time for each of the set of participants after each has initiated a change to a lifestyle characteristic associated with the set of lifestyle experiments; for each participant, determining a difference between the first spectral data and the second spectral data; and for each of the set of lifestyle experiments, based on the difference in spectral data for the set of participants, defining a health component channel comprising a weighted combination of spectral data from a set of wavelengths.

Clause 21. The method of clause 20, wherein the set of wavelengths is selected based on whether the wavelengths exhibit a unidirectional change in absorbance between the first spectral data and the second spectral data and which exhibit the unidirectional change for a majority of the participants.

Clause 22. The method of clause 20 or 21, further comprising, for each of the participants, identifying the set of wavelengths in the corresponding spectral data for the participant.

Clause 23. The method of clause 20, 21, or 22, further comprising constructing the health component channel by combining values from the set of wavelengths from the difference in spectral data for each of the set of participants.

Clause 24. The method of any of clauses 20-23, further comprising associating each of the set of participants with one or more demographic characteristics.

Clause 25. The method of any of clauses 20-24, wherein the health component channel is generated without determining a level of biomarker.

Clause 26. The method of any of clauses 20-25, wherein the set of wavelengths is selected by applying a form of principal components analysis to the set of difference of spectral data.

Clause 27. The method of any of clauses 20-26, wherein the health component channel of a biomatrix sample from a person is evaluated using a Z-score.

Clause 28. The method of any of clauses 20-27, further comprising associating a specific experiment with a biomatrix response pattern, wherein the bold response pattern represents an expected response of a person's biomatrix spectra to the person participating in the experiment.

Clause 29. The method of clause 28, wherein the biomatrix response pattern is determined based on the set of health component channels associated with a set of lifestyle experiments.

Clause 30. The method of clause 28 or 29, further compromising: determining a magnitude of the person's biomatrix response pattern; and displaying a trend of the magnitude over time to the person.

Clause 31. The method of clause 13, wherein the ranking comprises forming an ensemble of the ranking associated with each of two or more of the measures or data listed.

Clause 32. The method of any one of the preceding clauses, wherein the sample from the biomatrix comprises one of more of a urine sample, a saliva sample, a tear (lacrimal fluid) sample, an interstitial fluid sample, a sweat sample, or a fecal sample

Clause 33. An apparatus comprising: a processor configured to perform the method of any one of the preceding clauses; and a display to present a plurality of selected experiments as recommended experiments to the person.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A method of recommending a lifestyle experiment to a person, the method comprising:
obtaining spectral data from a sample of the person's biomatrix;
obtaining demographic characteristics for the person;
processing the spectral data to generate correlations for a set of health component channels associated with a set of lifestyle experiments;
generating a list of experiments from the set of lifestyle experiments for users with similar demographic characteristics;
comparing the correlations for the set of health component channels to the list of experiments to determine overlap between the correlations and the list; and
based on the comparison, selecting the lifestyle experiment from the set of lifestyle experiments as a recommended lifestyle experiment for the person.

2. The method of claim 1, wherein a health component channel of the set of health component channels is identified without identifying a biomarker in the biomatrix sample.

3. The method of claim 1, wherein the lifestyle experiment is selected from the set of lifestyle experiments based on a correlation between a health component channel of the experiment and the spectral data from the sample of the person's biomatrix.

4. The method of claim 3, wherein a plurality of lifestyle experiments is selected from the set of lifestyle experiments based on correlations between corresponding health component channels of each of the plurality of lifestyle experiments and the sample of the person's biomatrix.

5. The method of claim 4, wherein the plurality of lifestyle experiments is presented to the person for the person to select a lifestyle experiment from the plurality of lifestyle experiments.

6. The method of claim 1, wherein each member of the set of health component channels is associated with an experiment.

7. The method of claim 6, wherein the set of health component channels is defined at least in part by a weight probability matrix, the weight probability matrix comprising a plurality of weights to be combined with a plurality of values of the spectral data, each of the plurality of values of the spectral data associated with a wavelength of the spectral data.

8. The method of claim 7, wherein the spectral data of the person comprises a vector of spectral values and wherein combining the weight probability matrix with the vector of spectral values generates a correlation vector, the correlation vector comprising a set of correlations associated with the set of experiments.

9. The method of claim 8, wherein the lifestyle experiment is selected from the set of lifestyle experiments based on a corresponding value in the correlation vector.

10. The method of claim 1, wherein the demographic characteristics comprise one or more of age range, gender, race, location, highest education level, socioeconomic class, or nationality.

11. The method of claim 1, wherein the demographic characteristics for the person are used to generate a ranked list of experiments from users having similar demographics.

12. The method of claim 11, wherein a ranked experiment recommendation list is generated from an overlap between the ranked list of experiments and a correlation vector, the correlation vector comprising correlations between corresponding health component channels of each of the set of lifestyle experiments and the sample of the person's biomatrix.

13. The method of claim 11, wherein the ranked list of experiments is generated based on one or more of a measure of an amount of success each similar user had when they participated in an experiment, a number of people with a similar demographic characteristic who participated in an experiment, a measure of a deviation of a new user's biomatrix spectra from an initial or final biomatrix spectra of participants in an experiment, or how effective an experiment was at improving health of people having a similar demographic characteristic.

14. The method of claim 1, wherein the lifestyle experiment corresponds to a change a lifestyle characteristic comprising one or more of diet, exercise, drug consumption, alcohol consumption, or use of nicotine.

15. The method of claim 1, wherein each member of the set of health component channels comprises a weighting vector configured to combine spectral data from each of a plurality of wavelengths.

16. The method of claim 15, wherein said each member of the set of health component channels has been associated with demographic characteristics of prior users to generate the list of experiments based on demographic similarity between the prior users and the person.

17. The method of claim 1, wherein the spectral data is obtained by an infrared spectrometry system measuring a plurality of wavelengths of the sample of the person's biomatrix within a range from about 1000 nm to about 2000 nm, and wherein the spectral data from the sample of the person's biomatrix comprises from about 100 to about 500 intensity values for the plurality of wavelengths.

18. The method of claim 17, wherein the intensity values for the plurality of wavelengths are combined in accordance with a weighting function to determine an intensity value of an identified health component channel.

19. The method of claim 1, wherein a health component channel comprises a weighted combination of a plurality of values of the spectra of the biomatrix sample at a plurality of specific wavelengths.

20. A method of generating a dataset to process spectrometer data from biomatrix samples, the method comprising:
defining a set of lifestyle experiments associated with a change to a lifestyle characteristic;
selecting a set of participants to engage in the set of lifestyle experiments;
acquiring first spectral data from a first biomatrix sample for each of the set of participants at a first time to establish a baseline;
acquiring second spectral data from a second biomatrix sample at a second time for each of the set of participants after each has initiated a change to a lifestyle characteristic associated with the set of lifestyle experiments;
for each participant, determining a difference between the first spectral data and the second spectral data; and
for each of the set of lifestyle experiments, based on the difference in spectral data for the set of participants, defining a health component channel comprising a weighted combination of spectral data from a set of wavelengths.

21.-33. (canceled)
